(19) United States
(12) Patent Application Publication
(10) Pub. No.: US 2024/0310288 A1
(43) Pub. Date: Sep. 19, 2024

(54) TETRAZOLIUM-BASED COLORIMETRIC ASSAY

(71) Applicants: UNIVERSITY OF SOUTH CAROLINA, COLUMBIA, SC (US); CENTRO NACIONAL DE PESQUISA EM ENERGIA E MATERIAIS – CNPEM, Campinas- SP (BR)

(72) Inventors: EDWARD L. D'ANTONIO, BLUFFTON, SC (US); GUSTAVO F. MERCALDI, CAMPINAS- SP (BR)

(21) Appl. No.: 18/601,112

(22) Filed: Mar. 11, 2024

Related U.S. Application Data
(60) Provisional application No. 63/490,084, filed on Mar. 14, 2023.

(51) Int. Cl. G01N 21/78; C12N 9/99; C12Q 1/32; C12Q 1/34; C12Q 1/48

(52) U.S. Cl. CPC G01N 21/78; C12N 9/99; C12Q 1/32; C12Q 1/34; C12Q 1/485; G01N 2333/914

(57) ABSTRACT

Colorimetric based high-throughput assay systems are described that utilize colorimetric agents that exhibit both high water solubility characteristics in both an oxidized and a reduced state and exhibit intense UV/vis absorption in only one of the oxidized or reduced states, with absorption in the other state being either non-existent or very low. Disclosed assays are useful in examining the efficacy of enzyme inhibitors, including helicase or kinase inhibitors.

### CROSS REFERENCE TO RELATED APPLICATION

This application claims filing benefit of U.S. Provisional Patent Application Ser. No. 63/490,084 having a filing date of Mar. 14, 2023, which is incorporated herein by reference for all purposes.

### SEQUENCE LISTING

The instant application contains a Sequence Listing which has been submitted electronically in XML format and is hereby incorporated by reference in its entirety. Said XML copy, created on Feb. 29, 2024, is named USC-686_1525_SL.xml and is 6,096 bytes in size.

### BACKGROUND

The early treatment of disease can increase the curability of the disease, prevent long-lasting damage due to the disease, and reduce health care costs. For instance, the COVID-19 outbreak in 2019 resulted in great devastation across the globe, and still presents great danger to those in high-risk groups, primarily due to limited treatment options. A key starting point for early-stage drug discovery is to focus on finding inhibitors of critically important enzymes of the pathogen and/or disease progression.

COVID-19 is caused by a betacoronavirus known as SARS-CoV-2, which is a positive-sense single-stranded RNA (ssRNA) virus that has essential proteins for cell surface attachment, RNA and viral genomic replication, and translation of viral structural proteins. Once expressed in the host cell, 16 non-structural proteins (nsp) are produced that represent a drug discovery targeting opportunity. One target that has received attention in early-stage drug discovery is Coronavirus non-structural protein 13 (nsp13) known as SARS-CoV-2 helicase (SC2Hel). SC2Hel serves the function of unravelling double-stranded RNA (dsRNA) or double-stranded DNA (dsDNA) in an NTP-dependent method from the 5′ to 3′ directionality. SC2Hel is highly conservative with respect to the SARS-CoV helicase (SC1Hel), with a 99.8% protein sequence identity observed (100% sequence similarity) between the two helicases, in which one amino acid differs out of 601 amino acids.

The standard nucleic acid unwinding assay involving helicase from studies of SARS-CoV and SARS-CoV-2 utilizes the FRET-based fluorescence-quenching approach to display nucleic acid strand separation that is catalyzed by the helicase. The substrate structure specificity requires a ‘forked’ model or 5′-end single-stranded overhang that is at least 10 bases in length. This nucleic acid substrate generally has one strand containing a Cy3 fluorophore positioned at the 5′ end (Cy3 strand found on both DNA and RNA) and the opposing strand containing a Black Hole Quencher-2 (BHQ-2 found on DNA) or an Iowa black RQ quencher (AbRQ found on RNA) at the 3′ end (quencher strand). In the assay, a DNA competitor strand that is complementary to the Cy3 strand is employed to prevent the reannealing process of the substrate.

Other assay approaches have focused on SC2Hel NTPase activity as SC2Hel can be effectively driven by ATP. SC2Hel has an ATP-coupling stoichiometry of one ATP needed for each base pair (bp) unwound. NTPase activity assays have been performed using several methods. One method involves monitoring the reaction products due to helicase activity, in which the products are separated by thin-layer chromatography and observed by autoradiography through visual inspection. For instance, in one approach γ ³²P-ATP hydrolysis was quantitated as amount of inorganic phosphate (Pᵢ) was released and plotted against reaction time. Another NTPase assay involves the complexation of the released Pᵢ (from the NTP by helicase) with malachite green and molybdate (AM/MG reagent), which is a colorimetric method.

Many different colorimetric methods have been devised. For instance, the resazurin/resorufin system is commonly used in fluorometric methods due to the high fluorometric emission response of resorufin. Although resazurin (oxidized form) or resorufin (reduced form) can be used as UV/visible spectroscopy chromophores, this system is not ideal because both the oxidized form (also known as Alamar Blue™, with λₘₐₓ=603 nm) and the reduced form (λₘₐₓ=570 nm) both exhibit intense color absorption.

While the above represents improvement in the art, room for further improvement exists. What is needed in the art is a high throughput screening assay that can be utilized for discovery of inhibitors as may be used treatment of disease, including viral diseases such as COVID-19. In particular, an assay system utilizing a colorimetric reagent that transforms between a colorless or weakly colored form and an intense-colored form would be of great benefit to the art.

### SUMMARY

According to one embodiment, disclosed is a screening method for determining the efficacy of an enzyme inhibitor candidate. A method can include forming an assay composition. The assay composition can include the enzyme inhibitor candidate, the enzyme of interest, a substrate for the enzyme of interest, an electron transfer mediator, and a tetrazolium compound. The tetrazolium compound is one that is water soluble in both the reduced and oxidized states and that exhibits little or no absorbance in the UV/vis spectrum in one of the reduced or oxidized state while being highly absorbent in the UV/vis spectrum in the other state. A method can also include detecting a change in color in the assay composition upon reduction or oxidation of the tetrazolium compound. A detected change in color indicates the effectiveness of the candidate enzyme inhibitor with regard to inhibiting the activity of the enzyme of interest for the provided substrate.

Also disclosed is a high-throughput screening assay. The assay can include an enzyme, a substrate for the enzyme, and a tetrazolium compound that is water soluble in both the reduced and oxidized states and that exhibits little or no absorbance in the UV/vis spectrum in one of the reduced or oxidized state while being highly absorbent in the UV/vis spectrum in the other state. In one embodiment, the high-throughput screening assay can be provided in the form of an assay kit.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 provides a generic description of one embodiment of an enzyme-based colorimetric assay for a helicase inhibitor.

FIG. 2 provides a generic description of another embodiment of an enzyme-based colorimetric assay for a kinase inhibitor.

FIG. 3 provides at (a) the general structure of a 5′ single-stranded overhang 31/18-mer duplex nucleic acid substrate of SC2Hel, at (b) is provided SEQ ID NO: 1 and SEQ ID NO: 2 of the 31/18-mer dsDNA substrate of SC2Hel, at (c) is provided SEQ ID NO: 3 and SEQ ID NO: 4 of the 31/18-mer dsRNA substrate of SC2Hel.

FIG. 4 provides a description of one embodiment of a SARS-CoV-2 helicase enzyme-coupled colorimetric-based assay as described herein utilizing iodonitrotetrazolium chloride (INT) as the colorimetric reagent and the dsDNA substrate of FIG. 3 (b).

FIG. 5 schematically illustrates the SARS-CoV-2 helicase enzyme-coupled colorimetric-based assay of FIG. 4

FIG. 6 provides the structure of the oxidized form (a) and the reduced (b) form of INT. The reduced form (INT-formazan) has a strong absorption of visible light at λₘₐₓ=505 nm.

FIG. 7 provides the structure of the oxidized form (a) and the reduced (b) form of water-soluble tetrazolium salt-1 (WST-1). The reduced form (WST-1-formazan) has a strong absorption of visible light at λₘₐₓ=440 nm.

FIG. 8 provides the structure of the oxidized form (a) and the reduced (b) form of water-soluble tetrazolium salt-3 (WST-3). The reduced form (WST-3-formazan) has a strong absorption of visible light at λₘₐₓ=430 nm.

FIG. 9 provides the structure of the oxidized form (a) and the reduced (b) form of water-soluble tetrazolium salt-8 (WST-8). The reduced form (WST-8-formazan) has a strong absorption of visible light at λₘₐₓ=460 nm.

FIG. 10 presents UV-visible absorption spectra for the formation of INT-formazan (λₘₐₓ=505 nm) during a SC2Hel reaction kinetics experiment at various time points.

FIG. 11 provides a plot of [INT-formazan] vs. time (solid line) for the system.

FIG. 12 provides a Michaelis-Menten plot of the SC2Hel/INT assay showing the formation rate of INT-formazan as a function of dsDNA concentration.

FIG. 13 provides a Michaelis-Menten plot of the SC2Hel/INT assay showing the formation rate of INT-formazan as a function of ATP concentration.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

In general, the present disclosure is directed to colorimetric based high-throughput assay systems and methods for using the systems. The system utilizes colorimetric agents that exhibit both high water solubility characteristics and intense UV/vis absorption in only one of the oxidized or reduced states, with absorption in the other state being either non-existent or very low. As such, a reaction step in the assay (e.g., the final reaction step) can produce (or fail to produce) a water-soluble colorimetric agent, the presence or absence of which can be easily observed.

More specifically, disclosed assay systems and methods utilize tetrazolium compounds that exhibit water solubility in both the oxidized and reduced states. For instance, a tetrazolium compound can be fully dissolved in water at a concentration of 4.0 mM in either the oxidized or reduced states. In addition, the tetrazolium compounds exhibit little or no color absorption in one of the oxidized or reduced states, and high color absorption in the other oxidized or reduced state. For instance, when using a microplate reader in absorbance mode and considering the λₘₐₓ of the compound, when the lowest compound concentration measured is 0.0010 mM and the maximal compound concentration measured is 1.0 mM, then the compound can exhibit an absorbance value that is easily detectable in one of the oxidized or reduced states, e.g., 1,000 relative A.U. or greater, about 2,000 relative A.U. or greater, about 3,000 relative A.U. or greater, or about 5,000 relative A.U. or greater in some embodiments. For the other oxidized or reduced state that does not exhibit the high color absorption for the same λₘₐₓ of the original chromophoric compound, the compound can exhibit an absorbance value at λₘₐₓ of less than 1,000 relative A.U., such as about 700 relative A.U. or less, about 500 relative A.U. or less, or about 400 relative A.U. or less in some embodiments.

The tetrazolium colorimetric indicators of disclosed assay systems can show improvement over previously known colorimetric indicators. For instance, a 3-[4,5-dimethylthiazole-2-yl]-2,5-diphenyltetrazolium bromide (MTT)/MTT-formazan system has been previously utilized to evaluate cell viability. However, while MTT is water soluble, the assay involves conversion of MTT into a water-insoluble MTT-formazan compound. In contrast, disclosed assays utilize colorimetric agents based upon tetrazolium compounds that are water soluble in both the reduced and oxidized states.

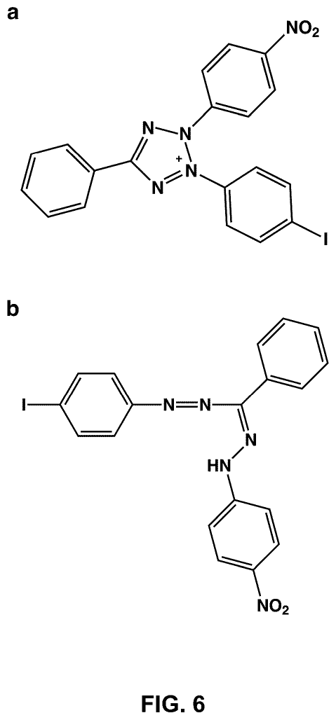

In one embodiment, the colorimetric agent can include iodonitrotetrazolium chloride (2-(4-Iodophenyl)-3-(4-nitrophenyl)-5-phenyl-2H-tetrazolium chloride, also commonly referred to as p-Iodonitrotetrazolium Violet, or INT; CAS No. 146-68-9) the structure of which is illustrated in FIG. 6. INT (oxidized), shown in FIG. 6 at (a) is essentially colorless in the visible region. When reduced to INT-formazan, as shown in FIG. 6 at (b), it exhibits an intense pink/purplish color with λₘₐₓ=505 nm.

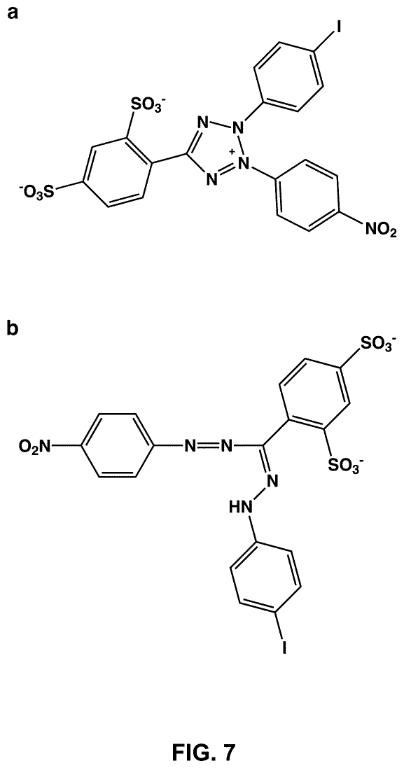

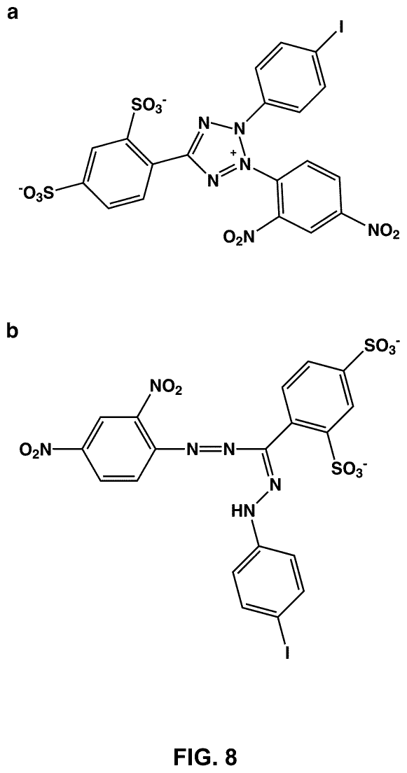

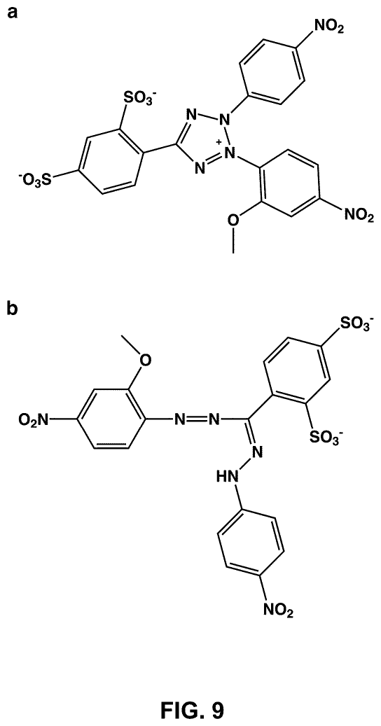

The colorimetric agent is not limited to INT, and other tetrazolium compounds that are water soluble in both the oxidized and reduced state and that exhibit little color absorbance in one state and strong color absorbance in the other are encompassed herein. By way of example, water soluble tetrazolium 1 (WST-1; CAS No. 150849-52-8), water soluble tetrazolium 3 (WST-3; CAS No. 515111-36-1), and water-soluble tetrazolium 8 (WST-8; CAS No. 193149-74-5) are examples of tetrazolium compounds useful in disclosed assays. The structure of WST-1 is shown in FIG. 7 in the oxidized (a) and reduced (b) states. When reduced to WST-1-formazan, it exhibits intense absorption with λₘₐₓ=440 nm. The structure of WST-3 is shown in FIG. 8 in the oxidized (a) and reduced (b) states. When reduced to WST-3-formazan, it exhibits intense absorption with λₘₐₓ=430 nm. The structure of WST-8 is shown in FIG. 9 in the oxidized (a) and reduced (b) states. When reduced to WST-8-formazan, it exhibits intense absorption with λₘₐₓ=460 nm.

During use in an assay protocol, the colorimetric agent can be utilized to determine the efficacy of a candidate enzyme inhibitor. For instance, in an assay protocol in which the candidate is effective at inhibiting the substrate interaction of an enzyme of interest, the tetrazolium compound can remain in the initial state, and no color change will occur. In contrast, in an assay protocol in which the candidate is not effective at inhibiting the enzyme/substrate interaction, the tetrazolium compound can be oxidized or reduced, leading to a well-defined and easily recognizable color change in the system. Of course, an assay protocol can follow the opposite course as well, i.e., in some embodiments, when the candidate is effective at inhibiting an enzyme/substrate interaction, the tetrazolium compound can be oxidized or reduced, leading to a well-defined color change in the assay mixture, and when the candidate is not effective as an inhibitor, the tetrazolium compound can remain in the initial state, with no significant color change in the assay mixture.

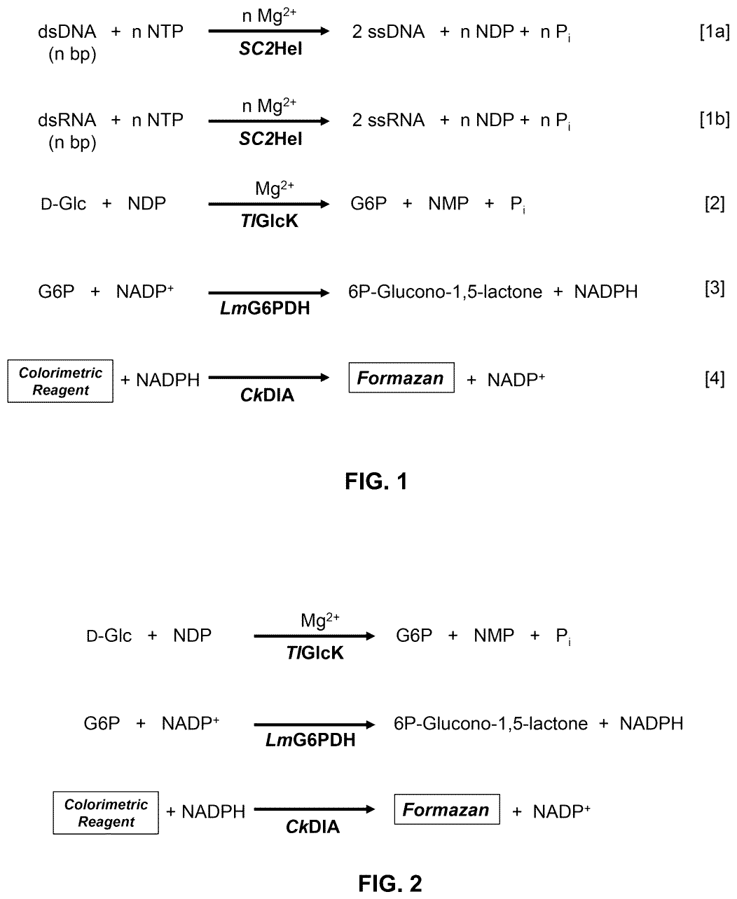

Inhibitor candidates as may be examined by use of an assay system can be designed for inhibition of any enzyme of choice. In one embodiment, an assay system can be utilized in examination of candidate helicase inhibitors, and in one particular embodiment in examination of candidate inhibitors of the SARS-CoV-2 nsp13 helicase (SC2Hel). FIG. 1 provides a general design for an assay protocol as may be used in examination of a candidate inhibitor of SC2Hel. As indicated, the colorimetric agent can be active in the final step of the protocol. In this particular embodiment, the colorimetric tetrazolium agent in the oxidized state is reduced to produce the formazan product and the electron transfer mediator (NADPH in this embodiment) is oxidized when the SC2Hel enzyme activity has not been inhibited by the candidate.

In the embodiment of FIG. 1, the formazan reaction is mediated by use of a diaphorase mediator, and, specifically by use of Clostridium kluyveri diaphorase (CkDIA). CkDIA can be beneficial in some embodiments, as tetrazolium compounds are fully compatible with CkDIA in producing the formazan product. In other embodiments, the formazan reaction can be mediated by use of an electron transfer mediator, in addition to or in place of a diaphorase mediator. For instance, the formazan reaction can be mediated by use of an electron transfer mediator such as phenazine methosulfate (PMS) and phenazine ethosulfate (PES) in tetrazolium dye-linked conversions to formazans.

While disclosed assay reagents can be particularly beneficial in discovery of helicase inhibitors, potential inhibitors of other enzyme/substrate interactions can also be examined by use of disclosed assays. For instance, in one embodiment, an assay can be utilized in discovery of kinase inhibitors. One embodiment of a kinase inhibitor assay protocol is shown in FIG. 2. In this particular embodiment, the enzyme of interest in the assay is a Thermococcus litoralis glucokinase (TlGlcK), which is an ADP-dependent glucose kinase. However, it should be understood that an assay is not limited to examination of any particular kinase.

In addition to the tetrazolium colorimetric agent, the candidate enzyme inhibitor, and the enzyme of interest, an assay will include a substrate for the enzyme of interest. A substrate can include, without limitation, a polynucleotide (e.g., a ssDNA, a ssRNA, a dsDNA, a dsRNA), a polypeptide (e.g., a protein substrate or a fragment thereof that includes a recognition motif for the enzyme), a carbohydrate, a glycoprotein, etc.

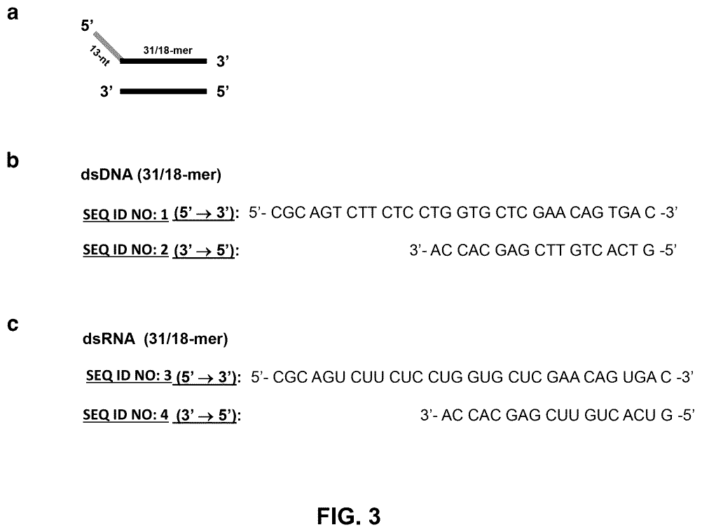

In those embodiments in which an assay is utilized in examination of a helicase inhibitor, the substrate can include dsRNA or dsDNA that is recognizable and unwound by the helicase of interest. FIG. 3 illustrates one embodiment of double stranded nucleic acid substrates as may be utilized in a helicase-based assay. As can be seen at (a) the generic structure of the substrate can include a 5′ overhang on one strand that is 13 bases in length, with the hybridized portion being 18 bases in length.

At (b) and (c) of FIG. 3 are provided specific substrate sequences that can be incorporated in a helicase-based assay, with the dsDNA substrate including a first strand as defined by SEQ ID NO: 1 and a second strand as defined by SEQ ID NO: 2 and the dsRNA substrate including a first strand as defined by SEQ ID NO: 3 and a second strand as defined by SEQ ID NO: 4. Of course, a helicase substrate is not limited to these particular substrates, and other dsRNA and dsDNA as are known may alternatively be utilized.

Other substrates for other enzymes of interest can likewise be incorporated in an assay. For instance, in those embodiments in which an assay is designed to examine candidate kinase inhibitors, the substrate can be a known substrate for the kinase of interest. In general, a kinase-based assay can include a phosphate donor, e.g., ATP or ADP, and a phosphate acceptor. In the embodiment illustrated in FIG. 2, the phosphate acceptor is D-glucose (D-Glc), which is phosphorylated to form glucose-6-phosphate (G6P) in the presence of the glucokinase. Other substrates as are known could alternatively be utilized, e.g., a substrate peptide containing the kinase recognition motif for the kinase of interest.

The assay system can also include an electron transfer mediator, which in some embodiments as discussed above can also function as a phosphate donor. In general, electron transfer mediators and phosphate donors as are known in the art can be utilized including, and without limitation to, nicotinamide adenine dinucleotide (NAD⁺), NADH, nicotinamide adenine dinucleotide phosphate (NADP⁺), NADPH, flavin adenine dinucleotide (FAD), FADH₂, flavin mononucleotide (FMN), FMNH₂, Coenzyme A, Coenzyme Q, tryptophan tryptophylquinone (TTQ), pyrroloquinolinequinone (PQQ), adenosine triphosphate (ATP), guanosine triphosphate (GTP), cytidine triphosphate (CTP), thymidine triphosphate (TTP), uridine triphosphate (UTP), adenosine diphosphate (ADP), guanosine diphosphate (GDP), cytidine diphosphate (CDP), thymidine diphosphate (TDP), and uridine diphosphate (UDP), or any combination thereof. Preferred electron transfer mediators can depend on the specific characteristics of an assay, as will be understood by one skilled in the art. For instance, the glucokinase of the assay of FIG. 2 is an ADP-dependent glucokinase and as such, the electron transfer mediator can include ADP or NDP, which can also function as the phosphate donor in the enzyme-mediated reaction.

Likewise, when incorporating a diaphorase in the assay system as mediator for the reduction of the colorimetric agent, it is known that certain diaphorase (e.g., CkDIA as utilized in the assays of FIG. 1 and FIG. 2) work very efficiently with NADPH in carrying out a 2-electron reduction of a tetrazolium compound into a formazan product. As such, in such an embodiment, it may prove beneficial to incorporate an NADPH electron transfer mediator in the system.

The assay system can include supporting enzymes as may be useful in one or more steps of an assay protocol. By way of example, the helicase-based assay of FIG. 1. involves a set of three supporting enzymes which are coupled to the helicase reaction; and as a result, a total of four enzyme can be included in the assay system. The particular supporting enzymes of choice can depend upon the reactions of the assay. For instance, in the embodiments of FIG. 1 and FIG. 2, the final step is mediated by a diaphorase, as discussed above. In addition, in the embodiment of FIG. 1, the kinase-mediated step is a secondary step that is coupled to the primary helicase mediated reaction. Both the assay of FIG. 1 and the assay of FIG. 2 include a dehydrogenase that mediates the hydrogenation and reduction of NADP⁺ to NADPH and the oxidation of G6P to form the lactone.

The supporting enzyme(s) can be from any source. In some embodiments, a bacterial enzyme can be utilized. For instance, in the embodiment of FIG. 1, the bacterial Leuconostoc mesenteroides glucose-6-phosphate dehydrogenase (LmG6PDH) and the archaebacterial Thermococcus litoralis glucokinase are utilized. A bacterial supporting enzyme can be preferred in some embodiments as the bacterial enzyme can be less susceptible to inhibition when compared to the mammalian enzyme, which is the case for mammalian G6PDH as compared to bacterial G6PDH. In other embodiments, a supporting enzyme from a yeast or non-mammalian prokaryote source could be used. For instance evidence suggests that G6PDH belonging to yeast and prokaryotic sources exhibit low susceptibility to inhibition, and this feature could be beneficial in coupled-enzymatic assays that rely on G6PDH activity. Moreover, the production of bacterial enzymes through heterologous expression in E. coli render higher yields compared to eukaryotic proteins, thus offering a cost advantage for the use of bacterial supporting enzymes.

The assay can include additional materials as are generally known in the art. By way of example, in those embodiments in which the enzyme of interest and/or one or more supporting enzymes is a metalloenzyme that utilizes a metal ion as a cofactor, the assay can include a salt of the metal, e.g., a salt of iron, copper, magnesium, zinc, manganese, calcium, etc., or any combination thereof.

In one embodiment, the assay can include a buffer, for instance to ensure desired activity of the enzyme of interest, to ensure solubility of one or more components of the assay, to prevent degradation of enzyme activity, to encourage product purification, and the like. Exemplary buffers can include, without limitation, phosphate buffers, Tris-HCl, triethanolamine (TEA), 4-(2-hydroxyethyl)piperazine-1-ethanesulfonic acid (HEPES), 4-morpholinepropanesulfonic acid (MOPS), etc., and combinations thereof.

In one embodiment, an assay can be provided as a combination kit. As used herein, the terms “combination kit” or “kit” is intended to refer to one or more components of an assay mixture and/or any additional components that can be used to package, sell, market, deliver, and/or provide the assay components to a user. Such additional components include, but are not limited to, packaging, syringes, pipettes, blister packages, and the like. When one or more of the compounds of an assay are provided simultaneously in a kit, the combination kit can contain the compounds in a single package. When one or more of the compounds are not provided simultaneously, the combination kit can contain each component in any combination. The separate kit components can be contained in a single package or in separate packages within a single kit package.

By way of example, a kit can include the primary enzyme of interest (e.g., a helicase or a kinase), a substrate for the enzyme, and a tetrazolium colorimetric agent as described. In embodiments, the kit can also include one or more supporting enzymes, one or more cofactors, buffers, etc. In some embodiments, a kit can include only a portion of the components of an assay. For instance an assay kit may be provided that does not include one or more of the electron transfer mediator(s), cofactors, buffers, etc., though these components may be utilized in carrying out an assay protocol.

In some embodiments, a kit also includes instructions printed on or otherwise contained in a tangible medium of expression. The instructions can provide information regarding the content of the kit, safety information regarding components of the kit, information regarding the assay, indications for successful use of the kit, and/or instructions for carrying out an assay. In some embodiments, the instructions can provide directions and protocols for providing the assay.

To carry out an assay, an assay composition can be formed that includes the enzyme of interest, the substrate, the candidate inhibitor, the tetrazolium compound, and any supporting materials as necessary, e.g., supporting enzymes, cofactors, buffers, etc.

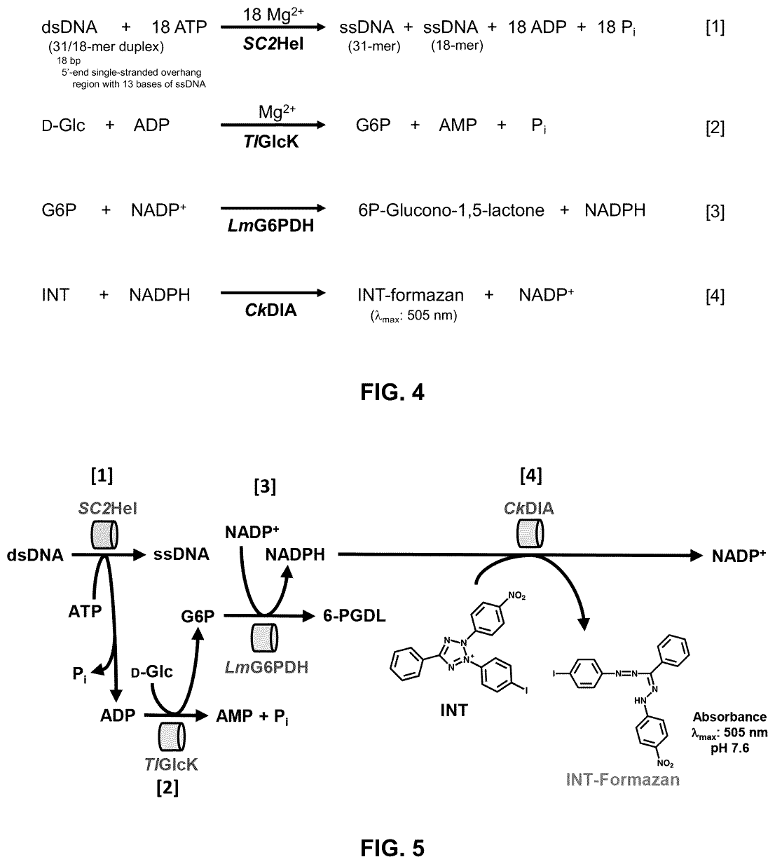

FIG. 4 and FIG. 5 illustrate one embodiment of an exemplary helicase assay. As shown, in the absence of an effective inhibitor, in step [1], the nucleic acid unwinding reaction of the dsDNA substrate (e.g., the dsDNA of FIG. 3 at (b)) is catalyzed by SC2Hel in the presence of 1 mol of ATP for every base pair (bp) of the substrate, as is known for the NTP-coupling stoichiometry for SC2Hel. Unwinding of the 31/18-mer dsDNA substrate by SARS-CoV-2 nsp13 helicase (SC2Hel) requires the energy of ATP and promotes the formation of ADP. The initial reaction of the assay can be initiated through addition of the ATP to the assay mixture or alternatively through addition of the enzyme to the assay mixture. As shown, the ADP formation is followed by coupling to INT-formazan formation through the ADP-dependent TlGlcK and NADP⁺-dependent LmG6PDH reactions.

In this embodiment, 1 mol of substrate (dsDNA or dsRNA) is used to produce 2 mol of converted product (ssRNA or ssDNA), ADP and inorganic phosphate (Pᵢ). In step [2], TlGlcK mediates conversion of 1 mol of ADP (from step [1]), 1 mol of D-Glc, and 1 mol of MgCl₂ to 1 mol of G6P, 1 mol of AMP, and 1 mol of Pᵢ. In step [3], LmG6PDH mediates conversion of 1 mol of G6P and 1 mol of NADP⁺ (both from step [2]) into 1 mol of 6-phospho-D-glucono-1,5-lactone and 1 mol of NADPH. Finally, in step [4], CkDIA converts 1 mol of the dissolved INT and 1 mol of NADPH (from step [3]) into 1 mol of NADP⁺ and 1 mol of INT-formazan.

In the presence of a candidate enzyme inhibitor that inhibits the initial helicase-mediated reaction of step [1], the colorimetric agent will not be converted and as such, the color of the assay mixture will not change. In contrast, in the presence of a candidate enzyme inhibitor that does not inhibit the initial helicase-mediated reaction of step [1], the colorimetric agent will be converted to the formazan product, and the assay mixture will exhibit a clear change in color.

The change in color can be simply observed or, in some embodiments, can be detected and recorded by use of a detector, e.g., through UV/vis spectrophotometry or the like.

The present invention may be better understood with reference to the examples set forth below.

### Example

### Materials

The 31/18-mer duplex dsDNA shown in FIG. 3 was purchased from Integrated DNA Technologies. Isopropyl β-D-thiogalactopyranoside (IPTG) was purchased from Carbosynth. Ethylenediaminetetraacetic acid tetrasodium salt hydrate, imidazole, bovine pancreas deoxyribonuclease I (DNase 1), bovine pancreas ribonuclease A (RNase A), triethanolamine, ρ3-nicotinamide adenine dinucleotide phosphate hydrate (NADP⁺), adenosine 5′-triphosphate disodium salt hydrate (ATP, >99%), iodonitrotetrazolium chloride, iodonitrotetrazolium violet-formazan, terrific broth, kanamycin sulfate, and dimethyl sulfoxide (DMSO) were purchased from Sigma (St. Louis, MO). Clostridium kluyveri diaphorase (CkDIA; E.C. 1.8.1.4) and Leuconostoc mesenteroides glucose 6-phosphate dehydrogenase (LmG6PDH; E.C. 1.1.1.49) were obtained from Worthington Biochemical Corporation (Lakewood, NJ). Luria-Bertani (LB) broth, lysozyme (type VI), cobalt-nitrolotriacetic acid (Co-NTA) resin, protease inhibitor tablets (EDTA-free), D-glucose, glycerol, magnesium chloride, sodium phosphate dibasic, potassium phosphate monobasic, DL-dithiothreitol, and all other chemicals were purchased from Fisher Scientific (Hampton, NH).

### Cloning

Gene synthesis was performed for the genes of Severe Acute Respiratory Syndrome Coronavirus 2 helicase nsp13 (GenBank accession number YP_009725308.1) and Thermococcus litoralis (DSM 5473) ADP-dependent glucokinase (GenBank accession number Q7M537.1) followed by cloning into separate kanamycin-resistant pET-28a(+) Escherichia coli expression vectors at restriction sites 5′ NcoI and 3′ HindIII at Azenta Life Sciences, Inc. (South Plainfield, NJ). Each construct encoded for an N-terminal hexahistidine tag (SEQ ID NO: 6), such as the segment MGRGSHHHHHHGMA (SEQ ID NO: 5) that preceded the start methionine. Codons for both plasmids were optimized to be used in protein expression of E. coli. The plasmid constructs were designated as pET-SC2Hel and pET-TlGlcK, respectively.

### Expression and Purification of Recombinant SC2Hel

Transformation of the plasmid pET-SC2Hel was performed by the heat shock method using E. coli strain BL21(DE3) (New England Biolabs) and colonies were grown on LB-agar plates with 50 μg/mL of kanamycin. Starter cultures containing 5 mL of LB broth, 50 μg/mL of kanamycin, and a single colony of E. coli from the transformation step were incubated at 37° C. and shaking at 250 rpm for 8 hr. using a New Brunswick Scientific C24 incubator shaker. The starter cultures were used to inoculate six 2 L culture flasks containing 500 mL of Terrific broth modified (Sigma) supplemented with 0.4% (v/v) glycerol and including 50 μg/mL of kanamycin that followed a 16 hr. incubation at 37° C. and shaking at 220 rpm. The culture flasks used autoclaved cheesecloth lids for the air flow. The 500 mL cultures were induced with IPTG to a final concentration of 1 mM and were incubated for an additional 24 hours at 28-32° C. with shaking at 220 rpm. The E. coli was centrifuged at 7,000 rpm where the resultant cell pellet was recovered and stored at −80° C., overnight. The frozen pellet was thawed from −80° C. to room temperature and resuspended in lysis buffer [50 mM HEPES (pH 7.0), 150 mM NaCl]. Cell lysis was performed by adding lysozyme and the cell suspension was stirred for 1 hr. at 4° C. before adding EDTA-free protease inhibitor tablets, after which the cell lysate was sonicated for 30 minutes in a water-bath sonicator (FS20, Fisher Scientific) and stored at −20° C. overnight. The cell lysate was thawed to room temperature and DNase I (Sigma) and RNase A (Sigma) at final concentrations of 10 μg/mL and 16 μg/mL, respectively, were added and stirred at 4° C. for 1 hr., followed by overnight freezing at −80° C. The cell lysate was thawed to room temperature and centrifuged at 15,000 rpm (Sorvall™ RC5C Plus centrifuge, SS-34 rotor) for 45 min at 4° C. and the resulting supernatant was recovered and loaded onto a cobalt-nitrilotriacetic acid (Co-NTA) immobilized-metal affinity chromatography column [1.5 cm (internal diameter)×4.0 cm (bed height)] that was pre-equilibrated with mobile phase A1 [50 mM HEPES (pH 7.0), 300 mM NaCl]. Mobile phase A1 was used as an initial wash to aid in the elution of most protein impurities. An isocratic step was applied from 0% to 13% mobile phase B1; note: mobile phase B1 [50 mM HEPES (pH 7.0), 300 mM NaCl, 150 mM imidazole]. After the UV absorbance (A=280 nm) on the chromatogram reached baseline, a gradient elution from 13-100% mobile phase B1 (50 mL gradient) was implemented in order to elute most of the other impurities from the column. Fractions of SC2Hel resulting from the Co-NTA step were pooled, concentrated using an Amicon™ Ultra YM-30 centrifugal concentrator (Millipore), and buffer exchanged into mobile phase A1 using a PD-10 desalting column (GE Healthcare). SC2Hel in mobile phase A1 was subjected to a nucleic acid digestion using DNAse I (purified from bovine pancreas that is free of any ribonucleases and proteases) and RNAse A (purified from bovine pancreas that is free of deoxyribonucleases and proteases) (Worthington Biochemical Corporation). Specifically, DNAse I (final concentration of 1.0 mg/mL) and RNAse A (final concentration of 1.0 mg/mL) were added to the pooled fractions SC2Hel at 4° C. for 3 hrs. The digested sample was immediately loaded onto a Co-NTA column [1.5 cm (internal diameter)×4.0 cm (bed height)] where the resin never used before. The resin was pre-equilibrated with mobile phase A1 (vide supra) and an isocratic step was applied from 0% to 100% mobile phase C1 [50 mM HEPES (pH 7.0), 1.0 M NaCl]. This MP-C1 (high salt concentration) wash step was implemented as a method to dissociate and elute off any interacting nucleic acids (DNA/RNA) with the helicase. A second isocratic step was applied in order to return to 100% MP-A1. Finally, a third isocratic step was applied to 100% MP-B1 to elute SC2Hel as nucleic acid free. Fractions of SC2Hel were pooled and concentrated to 5 mL using an Amicon Ultra YM-30 centrifugal concentrator (Millipore). The sample was loaded onto a 16/600 size-exclusion column (GE Healthcare) that was pre-equilibrated with filtered mobile phase A2 [50 mM triethanolamine (pH 7.6), 150 mM NaCl], which was the required buffer solution for the assay experiments (see below). Sodium dodecyl sulfate-polyacrylamide gel electrophoresis (SDS-PAGE) revealed the final fractions to be approximately 98% pure, by visual inspection. The pure fractions were pooled and concentrated to 0.16 mg/mL (2.34×10⁻⁶ mol/L) [E280=68,785 M⁻¹ cm⁻¹ (25); M.W.=68,425 g/mol (monomer of His-tagged SC2Hel)]. UV-visible spectrophotometry revealed an absorbance maximum at 280 nm (as opposed to 260 nm) for SC2Hel, indicating that the nucleic acid digestion was accomplished.

### Expression and Purification of Recombinant TlGlcK.

Transformation of the plasmid pET-TlGlcK was performed by the heat shock method using E. coli strain BL21(DE3) (New England Biolabs) and colonies were grown on LB-agar plates with 50 μg/mL of kanamycin. Starter cultures containing 5 mL of LB broth, 50 μg/mL of kanamycin, and a single colony of E. coli from the transformation step were incubated at 37° C. and shaking at 250 rpm for 8 hr. using a New Brunswick Scientific C24 incubator shaker. The starter cultures were used to inoculate six 2 L culture flasks containing 500 mL of Terrific broth modified (Sigma) supplemented with 0.4% (v/v) glycerol and including 50 μg/mL of kanamycin that followed a 16 hr. incubation at 37° C. and shaking at 220 rpm. The culture flasks used autoclaved cheesecloth lids for the air flow. The 500 mL cultures were induced with IPTG to a final concentration of 1 mM and were incubated for an additional 24 hrs. at 28° C.-32° C. with shaking at 220 rpm. The E. coli was centrifuged at 7,000 rpm where the resultant cell pellet was recovered and stored at −80° C. overnight. The frozen pellet was thawed from −80° C. to room temperature and resuspended in lysis buffer [50 mM 4-morpholinepropanesulfonic acid (MOPS) (pH 7.0), 150 mM NaCl]. Cell lysis was performed by adding lysozyme and the cell suspension was stirred for 1 hr. at 4° C. before adding EDTA-free protease inhibitor tablets, after which the cell lysate was sonicated for 30 minutes in a water-bath sonicator (FS20, Fisher Scientific) and stored at −20° C. overnight. The cell lysate was thawed to room temperature and DNase I (Sigma) and RNase A (Sigma) at final concentrations of 10 μg/mL and 16 μg/mL, respectively, were added and stirred at 4° C. for 1 hr., followed by overnight freezing at −80° C. The cell lysate was thawed to room temperature and centrifuged at 15,000 rpm (Sorvall RC5C Plus centrifuge, SS-34 rotor) for 45 min at 4° C. and the resulting supernatant was recovered and loaded onto a Co-NTA immobilized-metal affinity chromatography column [1.5 cm (internal diameter)×4.0 cm (bed height)] that was pre-equilibrated with mobile phase A1 [50 mM HEPES (pH 7.0), 300 mM NaCl]. Mobile phase A1 was used as an initial wash to aid in eluting protein impurities followed by an isocratic step to 13% mobile phase B1; note: mobile phase B1 [50 mM HEPES (pH 7.0), 300 mM NaCl, 150 mM imidazole]. After the UV absorbance (A=280 nm) on the chromatogram reached baseline, a gradient elution from 13%-100% mobile phase B1 (50 mL gradient) was implemented in order to elute most of the other impurities from the column. Fractions of TlGlcK resulting from the Co-NTA step were pooled and concentrated to 5 mL using an Amicon centrifugal concentrator (Millipore) equipped with a YM-30 membrane (30 kDa MWCO). The sample was loaded onto a 16/600 size-exclusion column (GE Healthcare) that was pre-equilibrated with filtered mobile phase A2 [50 mM triethanolamine (pH 7.6), 150 mM NaCl], which was the required buffer solution for the assay experiments (see below). SDS-PAGE revealed the final fractions to be >99% pure, by visual inspection. The pure fractions were pooled and concentrated to 1.0 mg/mL [E280=50,310 M⁻¹ cm⁻¹ (25); M.W.=55,192 g/mol (monomer of His-tagged TlGlcK)].

### Colorimetric-Based Nucleic Acid Unwinding Assay Using SC2Hel and INT

Observing enzymatic activity of SC2Hel in order to produce a Michaelis-Menten plot was performed by the following protocol. Briefly, a four-enzyme coupled colorimetric assay was performed as described in FIG. 4 and FIG. 5. The assay is based on the formation of INT-formazan at in aqueous solution, pH 7.6 (λₘₐₓ=505 nm). In the first step of the assay [1], SC2Hel in the presence of the overhang dsDNA substrate illustrated in FIG. 3 and ATP react to form a 31-oligomer ssDNA, an 18-oligomer ssDNA, and ADP. In the second step [2], TlGlcK in the presence of the formed ADP and D-glucose react to form G6P and AMP. In the third step [3], LmG6PDH in the presence of the formed G6P and NADP⁺ react to form 6-phospho-glucono-1,5-lactone and NADPH. Finally, in the fourth step [4], CkDIA in the presence of the formed NADPH and INT react to form NADP⁺ and INT-formazan. INT-formazan has a UV-visible spectrophotometric absorption at a wavelength maximum of 505 nm. All measurements were performed in triplicate.

In a given assay reaction mixture, the total volume was 168 μL. The following shows typical final assay concentrations (F.A.C.s) used in a given assay. F.A.C.s for enzymes: SC2Hel (0.89 μg/mL (or) 1.3×10⁻⁸ mol/L), TlGlcK (6.0 μg/mL), LmG6PDH (6.0 μg/mL), and CkDIA (0.6 U/mL). F.A.C.s for reagents: dsDNA substrate [31/18-mer dsDNA] (range: 0.625 μM-20.0 μM), assay buffer [50 mM triethanolamine (pH 7.6), 150 mM NaCl], D-glucose (1.9 mM), NADP⁺ (0.51 mM), MgCl₂ (7.8 mM), INT (0.60 mM), DMSO (1.0% by volume), and ATP (2.0 mM).

A typical reaction involved combining all enzyme solutions (vide supra) with all reagent solutions (vide supra) except that the reaction was started by adding ATP. Reactions were initiated with ATP, had a reaction time of 10.0 min, had a temperature of 22° C., and were run in the dark (left within the microplate reader after initiation). For quantification of INT-formazan, UV-visible spectrophotometric absorbance readings were recorded at 505 nm (λₘₐₓ) with a Tecan™ Spectrofluor Plus microplate reader in absorbance mode. A stock standard solution of 3.700 mM INT-formazan in 100% DMSO was prepared followed by mixing 25 μL into 175 μL of DMSO and performing a serial dilution, where each dilution divided the concentration by two. Each serial diluted standard was mixed with buffer [50 mM TEA (pH 7.6), 150 mM NaCl] as a 1:1 mixture to ensure an overall pH of 7.6. A standard curve was implemented for INT-formazan (relative absorbance vs. [INT-formazan]) at pH 7.6, in which the standard solutions of INT-formazan had a concentration range from 0.0018-0.2313 mM.

### Colorimetric-Based ATPase Assay Using SC2Hel and INT

The ATPase assay was performed in a similar manner to the nucleic acid unwinding assay (vide supra) except that the F.A.C. for the dsDNA substrate [31/18-mer dsDNA] was set to a value of 72.0 μM and ATP used a concentration range of 0.0156 mM-1.00 mM. Reactions were initiated with SC2Hel, had a reaction time of 10.0 min, had a temperature of 22° C., and were run in the dark (left within the microplate reader after initiation).

### Optimization of the Incubation Time and Quantity of SC2Hel for the Assay

To determine a satisfactory quantity of enzyme to use in the SC2Hel assay, absorption of INT-formazan at λₘₐₓ=505 nm was measured with an Agilent 8453 UV-visible spectrophotometer in the presence of various combinations of enzyme amounts (0.4, 2, 4, and 8 μg per reaction) and an incubation time course ranging from 32 s to 872 s at room temperature (22° C.). The assay protocol as described above was followed in order to carry out this determination (vide supra).

### Z′ Factor Determination

To determine the Z′ factor for the SC2Hel assay, there was an incubation time of 600 s, the assay was performed at room temperature, absorbance was monitored at 505 nm using the VANTAstar-F multimode microplate reader (BMG-Labtech), and all measurements were performed in septuplet. Furthermore, in a given assay reaction mixture for the positive controls, the following solution volumes and concentrations of reagents were added, as follows:
(a) 55.7 μL of Buffer A [50 mM TEA (pH 7.6), 150 mM NaCl],
(b) 29.3 μL of 10 mM D-glucose,
(c) 4.7 μL of 17 mM NADP⁺,
(d) 9.4 μL of 124 mM MgCl₂,
(e) 9.4 μL of 10 mM INT in: 100% DMSO,
(f) 20.0 μL of 0.40 mg/mL SC2Hel in: Buffer A,
(g) 0.94 μL of 1.0 mg/mL TlGlcK in: Buffer A,
(h) 0.94 μL of 1.0 mg/mL LmG6PDH in: Buffer A,
(i) 0.94 μL of 2.72 mg/mL diaphorase in: Buffer A,
(j) 18.8 μL of 160.0 μM dsDNA (31/18-mer) in: Buffer A, and
(k) 17.6 μL of 17 mM ATP.

In the case for preparing a given reaction mixture for a negative control (also performed in septuplet), the following solution volumes and concentrations of reagents were added, as follows:
(a) 94.4 μL of Buffer A [50 mM TEA (pH 7.6), 150 mM NaCl],
(b) 29.3 μL of 10 mM D-glucose,
(c) 4.7 μL of 17 mM NADP⁺,
(d) 9.4 μL of 124 mM MgCl₂,
(e) 9.4 μL of 10 mM INT in: 100% DMSO,
(f) 0.94 μL of 1.0 mg/mL TlGlcK in: Buffer A,
(g) 0.94 μL of 1.0 mg/mL LmG6PDH in: Buffer A,
(h) 0.94 μL of 2.72 mg/mL diaphorase in: Buffer A, and
(i) 17.6 μL of 17 mM ATP.

### Results

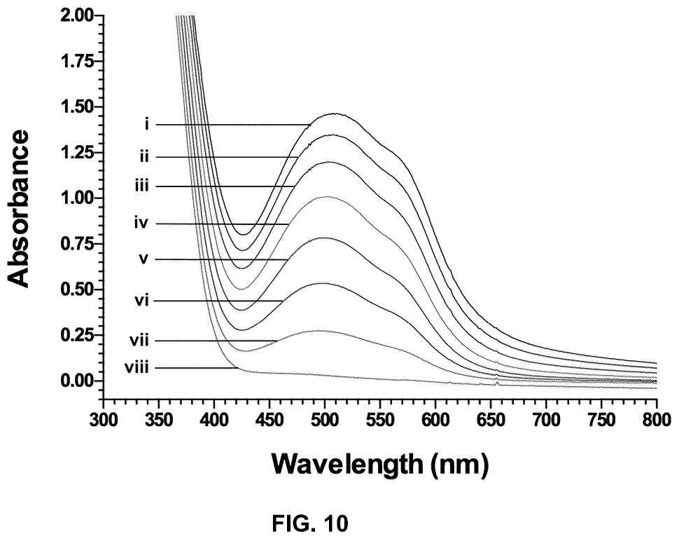

Kinetics of the SC2Hel/INT assay are shown in FIG. 10-FIG. 13. The UV-visible absorption spectra was monitored for INT-formazan production at various scanned time points [(i) 872 s, (ii) 752 s, (iii) 632 s, (iv) 512 s, (v) 392 s, (vi) 272 s, (vii) 152 s, and (viii) 32 s]. Results are shown in FIG. 10.

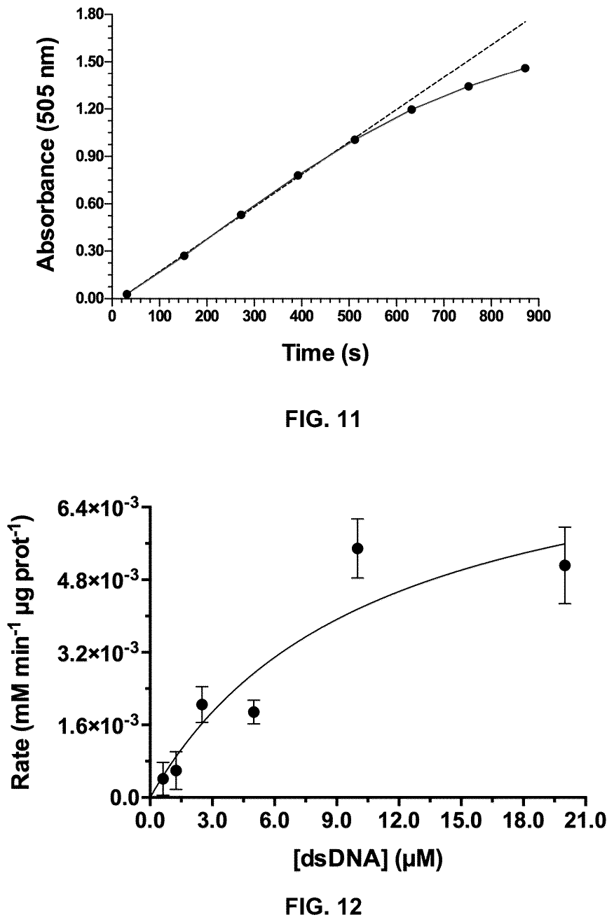

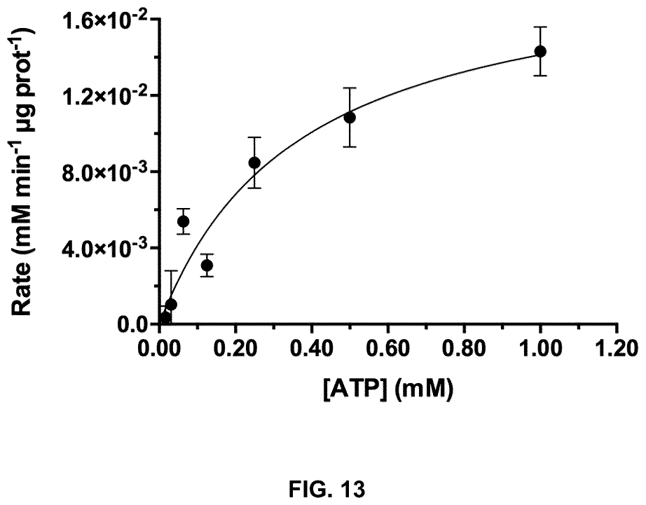

FIG. 11 shows the result of an analysis for the λₘₐₓ of INT-formazan (505 nm) as a function of time. A tangent line (dashed) was formed from the first five time points and was observed to run through data points ranging from 32 s to 512 s with a high R² value of 0.9995, after which the time trace slightly lost its linearity. The tangent line revealed a departure from the time trace plot and represented the linear regime of the reaction. The time point of 632 s was at a 5.9% deviation loss from the tangent line. Consequently, a slightly shorter time of 600 s was selected for the time optimum at room temperature to be within a 5% error. Through optimization of the incubation time concerning the linear regime of the enzymatic reaction, the steady-state activity was maintained and allowed for the observation of good Michaelis-Menten curves (FIG. 12, FIG. 13) and corresponding parameters (vide infra).

The Michaelis-Menten plot for the 31/18-mer dsDNA substrate is shown in FIG. 12. Results showed a K_{M} of 10.7±4.7 μM, a V_{MAX} of 8.571×10⁻³±1.938×10⁻³ mM min⁻¹ μg prot⁻¹, a k_{cat} of 560±190 min⁻¹, and a k_{cat}/K_{M} of 69±15 μM⁻¹ min⁻¹. For ATP (FIG. 13), results showed a K_{M} of 0.367±0.120 mM, a V_{MAX} of 1.932×10⁻²±2.872×10⁻³ mM min⁻¹ μg prot⁻¹, a k_{cat} of 1400±460 min⁻¹, and a k_{cat}/K_{M} of 4800±2800 mM⁻¹ min⁻¹. The mean and SD of three independent experiments are shown in both plots.

In obtaining FIG. 12, various concentrations of the 31/18-mer dsDNA substrate were used (range: 0.625-20.0 μM) and a constant concentration of ATP (2.00 mM). In obtaining FIG. 13, the assay used various concentrations of ATP (range: 0.0156-1.00 mM) and a constant concentration of 31/18-mer dsDNA substrate (72.0 μM). In all cases, the final assay concentration of SC2Hel was 1.3×10⁻⁸ mol/L, experiments were performed at 22° C., and the reaction time was 10.0 min. Data analysis and plots were prepared in GraphPad Prism.

In the control experiments where SC2Hel was not added in the reaction mixture there was a residual production of INT-formazan, which was attributed to the spontaneous degradation of ATP over time into ADP+Pᵢ. The ADP formed appears to have proceeded into reaction [2] of FIG. 4. In data processing, the mean absorbance signal observed from three of the controls was assessed and background subtracted to make a correction in the amount of INT-formazan produced from the helicase assay. In general, the residual production of INT-formazan was not extensive as very pure ATP was used. Aliquots of a prepared ATP solution (dissolved in deionized ultrapure water) were frozen at −80° C. and the working solutions were used immediately after being thawed out on ice.

To interrogate the assay's validation parameters, seven replicates of positive and negative controls were inspected. All samples were run at an incubation time of 600 s at room temperature. A signal-to-noise ratio and signal-to-background ratio of 439 and 294, respectively, were observed, which were very high and acceptable values. Additional assay parameters calculated included a signal window of 21.5, an assay variability ratio of 0.13, and a Z′ factor of 0.87. From the observation of positive and negative controls having exhibited very low variability, a high signal-to-noise, and a high Z′ factor, it was concluded that the assay demonstrated excellent performance characteristics. In particular, Z′ factor values close to the value of 1.0 and much greater than 0.5 is an important indicator for validating a screening assay.

Overall, the example illustrated that the disclosed colorimetric enzymatic assay successfully detected ADP resulting from the nucleic acid unwinding (using a 31/18-mer dsDNA substrate) and ATPase activities. This assay is advantageous as it is easier to employ compared to the traditional FRET-based helicase assay. Beneficially, disclosed assay systems can provide rapid, sensitive, and colorimetric results.

While certain embodiments of the disclosed subject matter have been described using specific terms, such description is for illustrative purposes only, and it is to be understood that changes and variations may be made without departing from the spirit or scope of the subject matter.

What is claimed is:

1. A screening method for determining the efficacy of an enzyme inhibitor candidate, comprising:
forming an assay composition, the assay composition comprising the enzyme inhibitor candidate, a first enzyme, a substrate of the first enzyme, an electron transfer mediator, and a tetrazolium compound, wherein the tetrazolium compound is water soluble in a reduced state and in an oxidized state, and wherein the tetrazolium compound exhibits little or no absorbance in the UV/vis spectrum in one of the reduced state and the oxidized state and exhibits strong absorbance in the UV/vis spectrum in the other of the reduced state and the oxidized state; and
detecting a change in color in the assay composition, wherein the color change indicates the effectiveness of the enzyme inhibitor candidate in inhibiting an activity of the first enzyme for the substrate of the first enzyme.

2. The screening method of claim 1, wherein the tetrazolium compound comprises iodonitrotetrazolium chloride, water soluble tetrazolium 1, water soluble tetrazolium 3, or water soluble tetrazolium 8.

3. The screening method of claim 1, wherein the first enzyme is a helicase.

4. The screening method of claim 3, wherein the helicase is a SARS-CoV-2 helicase.

5. The screening method of claim 1, wherein the first enzyme is a kinase.

6. The screening method of claim 1, wherein the substrate comprises a polynucleotide, a polypeptide, a carbohydrate, or a glycoprotein.

7. The screening method of claim 1, wherein the electron transfer mediator comprises ATP, ADP, NAD, NADH, NADP⁺, NADPH, FAD, FADH, FMN, FMNH, Coenzyme A, Coenzyme Q, tryptophan tryptophylquinone, pyrroloquinolinequinone, or any combination thereof.

8. The screening method of claim 1, wherein the assay composition comprises one or more supporting enzymes.

9. The screening method of claim 8, wherein the one or more supporting enzymes comprises a dehydrogenase, a kinase, a diaphorase, or any combination thereof.

10. The screening method of claim 8, wherein the one or more supporting enzymes comprises a bacterial enzyme, an archaebacterial enzyme, a yeast enzyme, or a non-mammalian prokaryote enzyme.

11. The screening method of claim 1, further comprising initiating the screening method by adding the electron transfer mediator as a final addition to the assay composition.

12. The screening method of claim 1, further comprising initiating the screening method by adding the first enzyme to the assay composition.

13. The screening method of claim 1, wherein the change in color is detected by use of UV/vis spectrophotometry.

14. An assay comprising:
a first enzyme;
a substrate of the first enzyme; and
a tetrazolium compound, wherein the tetrazolium compound is water soluble in a reduced state and in an oxidized state, and wherein the tetrazolium compound exhibits little or no absorbance in the UV/vis spectrum in one of the reduced state and the oxidized state and exhibits strong absorbance in the UV/vis spectrum in the other of the reduced state and the oxidized state.

15. The assay of claim 14, wherein the tetrazolium compound comprises iodonitrotetrazolium chloride, water soluble tetrazolium 1, water soluble tetrazolium 3, or water soluble tetrazolium 8.

16. The assay of claim 14, wherein the first enzyme is a helicase.

17. The assay of claim 16, wherein the helicase is a SARS-CoV-2 helicase.

18. The assay of claim 14, wherein the first enzyme is a kinase.

19. The assay of claim 14, wherein the substrate comprises a polynucleotide, a polypeptide, a carbohydrate, or a glycoprotein.

20. The assay of claim 14, further comprising one or more supporting enzymes.

21. The assay of claim 20, the one or more supporting enzymes comprising a dehydrogenase, a kinase, a diaphorase, or any combination thereof.

22. A kit comprising the assay of claim 14.


SEQUENCE LISTING (6 sequences)

SEQ ID NO: 1 - DNA, 31 bp, synthetic construct
  source 1..31: mol_type = other DNA
cgcagtcttctcctggtgctcgaacagtgac

SEQ ID NO: 2 - DNA, 18 bp, synthetic construct
  source 1..18: mol_type = other DNA
gtcactgttcgagcacca

SEQ ID NO: 3 - RNA, 31 nt, synthetic construct
  source 1..31: mol_type = other RNA
cgcagtcttctcctggtgctcgaacagtgac

SEQ ID NO: 4 - RNA, 18 nt, synthetic construct
  source 1..18: mol_type = other RNA
gtcactgttcgagcacca

SEQ ID NO: 5 - protein, 14 aa, synthetic construct
MGRGSHHHHHHGMA

SEQ ID NO: 6 - protein, 6 aa, synthetic construct
HHHHHH